(19) United States
(12) Patent Application Publication
(10) Pub. No.: US 2002/0064509 A1
(43) Pub. Date: May 30, 2002

(54) Method for producing a goniochromatic effect comprising applying to skin a cosmetic composition comprising at least one continuous lipophilic phase and at least one goniochromatic pigment

(76) Inventors: Sabine Grimm, Chatenay Malabry (FR); Jean-Christophe Simon, Paris (FR)

Correspondence Address: Thomas L. Irving, FINNEGAN, HENDERSON, FARABOW,, GARRETT & DUNNER, L.L.P., 1300 I Street, N.W., Washington, DC, 20005-3315, US

(21) Appl. No.: 09/968,967

(22) Filed: Oct. 3, 2001

(30) Foreign Application Priority Data
Oct. 3, 2000 (FR) 00 12600

(51) Int. Cl. A61K 7/021

(52) U.S. Cl. 424/63

(57) ABSTRACT

A cosmetic composition, for example a makeup composition, containing at least one continuous lipophilic phase, for example, a nonanhydrous phase, and at least one goniochromatic pigment. It also relates to the use of the composition in order to create a goniochromatic effect on the skin, the hair, the eyebrows, or the eyelashes.

The present invention relates to a novel method for producing a goniochromatic effect on skin of human beings comprising applying to the skin of human beings a cosmetic composition, in certain embodiments a makeup composition, comprising at least one continuous lipophilic phase and at least one goniochromatic pigment, wherein the at least one goniochromatic pigment has an interferential multilayer structure.

Makeup compositions, such as free powders, compact powders, foundations, blushers, eyeshadows, and lipsticks, may comprise an appropriate vehicle and various coloring agents intended to confer a certain color on the compositions, before and/or after their application to at least one of the skin (including the lips), and the superficial body growths.

A fairly restricted range of coloring agents, for example pigments, such as lakes, inorganic pigments, and pearlescent pigments, is currently used to create colors. Lakes make it possible to obtain vivid colors but are generally unstable to light, temperature, and pH. After application, some also stain the skin in an unsightly way, by discharge of the colorant. Inorganic pigments, for example inorganic oxides, are, in contrast, very stable but give rather dull and pale colors. Pearlescent pigments do not provide intense colors with an iridescent effect. Rather, pearlescent pigments make it possible to obtain varied colors with generally fairly weak color effects, for example, the color effect is mainly visible along only one given angle corresponding to a spectral reflection. For example, patent WO-A-96/03962 discloses a cosmetic composition in the form of an emulsion comprising a silicone oil, an “interferential” pigment of platelet type composed of a support such as mica coated with a given layer of titanium oxide with a given thickness, and a pigment based on iron oxide. Due to the interferential pigment present in the composition, the composition gives a shade along a given angle and does not produce a goniochromatic effect.

One of the objects of the present invention is to provide a cosmetic composition which overcomes at least one of the above-mentioned disadvantages. Another of the objects of the present invention is to provide a cosmetic composition which exhibits at least one of the following effects, for example aesthetic, goniochromatic, and volume effects. The cosmetic composition may also exhibit a soft-focus effect, i.e., an effect which may conceal the microreliefs of the skin (including the lips). Such effects may be obtained by use of compositions, for example, chosen from foundations, eye shadows, blushes, lipsticks, lip glosses, lip lacquers, mascaras, and eyeliners.

The invention thus relates to a method of producing at least a goniochromatic effect on skin of human beings, comprising applying to the skin of human beings a cosmetic composition comprising a cosmetically acceptable medium comprising (a) at least one continuous lipophilic phase, in certain embodiments a nonanhydrous phase, and (b) at least one goniochromatic pigment, wherein the at least one goniochromatic pigment has an interferential multilayer structure.

The goniochromatic effect is understood to mean that the hue of the skin of human beings may change around the circumference of the L*a*b* color space and that the lightness of the skin of human beings may increase or decrease along the L* axis of the L*a*b* color space. The L*a*b* color space and hue are described in “Precise Color Communication Color Control From Feeling to Instrumentation,” Minolta Co., Ltd, 1994, the disclosure of which is hereby incorporated by reference. The L* axis of the color space indicates lightness and the a* and b* are the chromaticity coordinates.

Once the composition is applied to the skin, the composition may produce different hues on the skin, for example red or green, depending upon the angle of the incident light reflecting off of the skin and the angle of observation. The hues may appear and disappear according to the movements of the person wearing the composition and the angle of the incident light. The skin of human beings thus appears to be “living,” thereby creating a volume effect. Moreover, certain features of the skin will appear lighter as compared to other features of the skin depending upon the angle of the incident of the light reflecting off of the skin and the angle of observation. For example, the cheek bones, a prominent feature of skin, may appear lighter than the fleshy part of the cheek, a nonprominent feature of skin, thereby making the cheeks look hollower.

The goniochromatic effect is achieved by the presence of the at least one goniochromatic pigment having an interferential multilayer structure in the cosmetic composition. The term “at least one goniochromatic pigment” may be understood to mean a goniochromatic pigment having an interferential multilayer structure. The “interferential multilayer structure” may be understood to mean at least a multilayer structure, in certain embodiments, at least a bilayer structure. The multilayers may make it possible to create changes in hue and lightness depending upon the angle of the incident light and how it is reflected and the angle of observation. For example, the incident light is reflected off of the multilayers of the at least one goniochromatic pigment at different angles thereby creating changes in hue and lightness also known as the goniochromatic effect defined above. These changes may also be noticed depending upon the angle of observation.

The interferential multilayer structure of the at least one goniochromatic pigment may comprise at least two layers, each layer may, independently of the other layers, be made of at least one material chosen from

MgF₂, CeF₃, ZnS, ZnSe, Si, SiO₂, Ge, Te, Fe₂O₃, FeO₃, Pt, V, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, TI, Ta, W, Zn, MoS₂, Cr, mica oxide, cryolite, alloys, polymers, and their combinations. The interferential multilayer structure may be inorganic or organic. Different hues are obtained according to the thickness of each of the various layers.

In certain embodiments, for example, the interferential multilayer structure of the at least one goniochromatic pigment may be chosen from the structures: Al/SiO₂/Al/SiO₂/Al; Cr/MgF₂/Al/MgF₂/Al; MoS₂/SiO₂/Al/SiO₂/MoS₂; Fe₂O₃/SiO₂/Al/SiO₂/FeO₃; Fe₂O₃/SiO₂/Fe₂O₃/SiO₂/Fe₂O₃; MoS₂/SiO₂/mica-oxide/SiO₂/MoS₂; and Fe₂O₃/SiO₂/mica-oxide/SiO₂/Fe₂O₃.

In some embodiments, the structure may be composed of alternating layers of materials having a low optical index and a high optical index.

The at least one goniochromatic pigment according to the invention, in certain embodiments, may be chosen from the following commercial goniochromatic pigments: INFINITE COLORS from Shiseido, SICOPEARL FANTASTICO from BASF, COLORSTREAM from Merck, XIRALLIC from Merck, COLORGLITTER from 3 M, and CHROMAFLAIR from Flex. Examples of the at least one goniochromatic pigment according to the invention and how to make them are described in U.S. Pat. No. 6,193,794, the disclosure of which is hereby incorporated by reference.

The at least one goniochromatic pigment according to the invention may be incorporated into at least one of a cosmetic and dermatological composition, for example a makeup composition, having at least one continuous lipophilic phase, in certain embodiments a nonanhydrous phase, in an amount which can be easily determined by a person skilled in the art on the basis of his general knowledge. For example, the at least one goniochromatic pigment may be present in the composition in an amount ranging from from 0.01 to 50%, in some embodiments from 0.5 to 25%, by weight with respect to the total weight of the composition. Even at a high concentration, the at least one goniochromatic pigment may have no destructuring effect on the composition.

The term “color” may be understood to mean any color in the visible spectrum.

The composition of the invention may be provided in the form of a product to be applied to at least one of the skin,, and the superficial body growths of human beings, in certain embodiments the skin, the hair, the lips, the eyelashes, or the eyebrows of human beings. The composition thus can comprise a cosmetically acceptable medium, i.e., a medium compatible with the skin, the hair, the lips, the eyelashes, and the eyebrows of human beings.

According to the invention, this medium may comprise or be provided, in certain embodiments, in a form chosen from a suspension in water, a dispersion in water, a solution in water, an aqueous/alcoholic medium which is optionally thickened, indeed even gelled; an oil-in-water (O/W) emulsion in a form chosen from a cream, a paste, and even a solid; a multiple (W/O/W) emulsion in a form chosen from a cream, a paste, and even a solid; aqueous gel; aqueous/alcoholic gel; hydrophilic foam; emulsified gel; dispersion of vesicles, for example a dispersion chosen from ionic lipids and nonionic lipids; two-phase lotion; multiphase lotion; and spray. A person skilled in the art may choose the appropriate pharmaceutical dosage form, and its method of preparation, on the basis of his/her general knowledge, taking into account, on the one hand, the nature of the constituents used, for example their solubility in the vehicle, and, on the other hand, the application envisaged for the composition. The composition may be, in certain embodiments, nonanhydrous, i.e., the composition is not devoid of water.

The composition thus can comprise said cosmetically acceptable medium, which comprises at least one continuous lipophilic phase, in certain embodiments at least one nonanhydrous phase, which can comprise at least one lipophilic substance chosen from (a) at least one fatty substance which is liquid at ambient temperature (generally 25° C.) and atmospheric pressure (760 mmHg generally, i.e. 1.013×10⁵ Pa), for example, chosen from at least one oil and at least one water-immiscible organic solvent, and (b) at least one fatty substance which is solid at ambient temperature and atmospheric pressure, for example chosen from waxes, gums, pasty fatty substances, and solid fatty substances. The at least one continuous lipophilic phase may be present in the composition in an amount ranging from 0.5 to 99.99% by weight with respect to the total weight of the composition.

When the medium is provided in the form of an emulsion, the composition according to the invention may optionally comprise, in addition, at least one surfactant. The at least one surfactant may be present in the composition in an amount up to 30%, in certain embodiments in an amount ranging from 0.01 to 30%, by weight with respect to the total weight of the composition.

According to the application envisaged, the composition may comprise, in addition, at least one film-forming polymer, for example chosen from polyurethanes, polyacrylics, polyurethane hybrids, polyacrylic hybrids, polyesters, nitrocellulose, hydrocarbonaceous resins, siliconeresins, and their mixtures. This is, in certain embodiments, the case when it is desired to prepare a composition of mascara or eyeliner type or a hair composition of lacquer type. The at least one film-forming polymer may be dissolved or dispersed in a cosmetically acceptable medium and may optionally be used in combination with at least one coalescence agent and/or at least one plasticizer.

The at least one fatty substance which is liquid at ambient temperature and at atmospheric pressure, for example at least one oil, which can be used in the invention may be chosen, for example, from hydrocarbonaceous oils of animal origin, such as perhydrosqualene; vegetable hydrocarbonaceous oils, such as liquid triglycerides of fatty acids comprising 4 to 10 carbon atoms, such as jojoba oil, karate butter oil, triglycerides of heptanoic acid, triglycerides of octanoic acid, sunflower oil, maize oil, soybean oil, grape seed oil, sesame oil, apricot oil, macadamia oil, castor oil, avocado oils, and triglycerides of caprylic/capric acids; linear and branched hydrocarbons of mineral and synthetic origin, such as liquid paraffins and their derivatives, liquid petrolatum, polydecenes, and hydrogenated polyisobutene, such as Parleam; isododecane; synthetic esters and ethers, for example of fatty acids, such as, for example, Purcellin oil, isopropyl myristate, 2-ethylhexyl palmitate, 2-octyldodecyl stearate, 2-octyldodecyl erucate, isostearyl isostearate, and isononyl isononanoate; hydroxylated esters, such as isostearyl lactate, octyl hydroxystearate, octyidodecyl hydroxystearate, diisostearyl malate, and triisocetyl citrate; heptanoates, octanoates of fatty alcohols, and decanoates of fatty alcohols; polyol esters, such as propylene glycol dioctanoate, neopentyl glycol diheptanoate, diethylene glycol diisononanoate and pentaerythritol esters; fatty alcohols having from 12 to 26 carbon atoms, such as octyidodecanol, 2-butyloctanol, 2-hexyldecanol, 2-undecylpentadecanol, and oleyl alcohol; fluorinated oils which are partially hydrocarbon-comprising and/or silicone-comprising; silicone oils, such as volatile or nonvolatile and linear and cyclic polydimethylsiloxanes (PDMSs) which are liquid or pasty at ambient temperature and at atmospheric pressure, such as cyclomethicones, dimethicones, optionally comprising a phenyl group, such as phenyl trimethicones, phenyltrimethylsiloxydiphenylsiloxanes, diphenylmethyldimethyltrisiloxanes, diphenyl dimethicones, phenyl dimethicones, and polymethylphenylsiloxanes; and their mixtures.

The at least one oil may be present in the at least one continuous lipophilic phase in an amount, for example up to 90%, in certain embodiments in an amount ranging from 0.01 to 85%, by weight with respect to the total weight of the at least one continuous lipophilic phase.

The composition of the invention may comprise at least one fatty phase which is solid or pasty at ambient temperature and at atmospheric pressure comprising at least one compound chosen from waxes, pasty fatty substances, and gums. The at least one wax, for example, may be chosen from hydrocarbonaceous, fluorinated waxes, and silicone waxes and may be at least one of vegetable origin, mineral origin, animal origin, and synthetic origin. In certain embodiments, the at least one wax may exhibit a melting temperature of greater than approximately 25° C., in other embodiments of greater than approximately 45° C., and the at least one pasty fatty substance may exhibit a melting temperature ranging from approximately 25° C. to approximately 45° C.

The at least one wax which may be used in the composition of the invention is, for example, chosen from beeswax, carnauba wax, candelilla wax, jojoba wax (hydrogenated and nonhydrogenated), paraffin wax, microcrystalline waxes, ceresin or ozokerite; synthetic waxes, such as polyethylene waxes and Fischer-Tropsch waxes; silicone waxes, such as alkyl dimethicones having from 16 to 45 carbon atoms and alkoxy dimethicones having from 16 to 45 carbon atoms.

The at least one gum which may be used according to the invention may generally be chosen from PDMSs with a high molecular weight, cellulose gums, polysaccharides, and their mixtures, and the pasty substance which may be used according to the invention may generally be chosen from hydrocarbonaceous compounds, such as lanolins and their derivatives, PDMSs, and their mixtures.

The nature and the amount of the solid substances present in the composition depend on the mechanical properties and textures desired. The at least one fatty substance which is solid at ambient temperature and at atmospheric pressure may be present in the composition in an amount, for example, up to 50%, in certain embodiments in an amount ranging from 0.01 to 40%, and in other embodiments in an amount ranging from 0.1 to 30%, by weight with respect to the total weight of the composition.

The composition of the invention may further comprise at least one additional particulate phase which may be present in the composition in an amount, for example, up to 50%, in certain embodiments in an amount ranging from 0.01 to 40%, and in other embodiments in an amount ranging from 0.05 to 30%, by weight with respect to the total weight of the composition. The composition may also comprise at least one pigment other than the at least one goniochromatic pigment according to the invention. Moreover, the composition may also comprise at least one pearlescent agent and at least one filler used conventionally in cosmetic compositions.

The at least one additional particulate phase may make it possible to better control the color path across the composition so as to avoid some hues which may appear as unsightly to some users (such as green, for example, for users of foundation under conventional conditions) or so as to enhance the range of hues provided to the users starting from the same at least one goniochromatic pigment. Moreover, the at least one additional particulate phase makes it possible to reduce the manufacturing cost of the cosmetic composition according to the invention as, currently, the cost of the at least one goniochromatic pigment according to the invention is high.

The term “at least one pigment other than the at least one goniochromatic pigment according to the invention” may be understood to mean white and colored and inorganic and organic particles which are insoluble in a liquid hydrophilic phase and which are intended to color and/or opacify the composition. Such pigments are generally monocolors. The term “pigment” may be understood to mean particles which are insoluble in the medium constituting the cosmetic composition, i.e., in the dispersed or solid state in one of the phases of said medium, and which may be used to create color, modify shades of color, and opacify said composition. In addition to pigments, such as lakes, pearlescent pigments, and inorganic pigments, there are also interferential pigments which may be used in compositions according to the present invention. The interferential pigment may be provided in the form of glitter, with a metallic color, for example, a substrate or core such as mica, with an applied coating of, for example, titanium dioxide.

Examples of the at least one pigment other than the at least one goniochromatic pigment, according to the invention, may be, in certain embodiments, those disclosed in the following documents: U.S. Pat. No. 3,438,96, EP-A-227 423, U.S. Pat. No. 5,135,812, EP-A-170 439, EP-A-341 002, U.S. Pat. No. 4,930,866, U.S. Pat. No. 5,641,719, EP-A-472 371, EP-A-395 410, EP-A-753 545, EP-A-768 343, EP-A-571 836, EP-A-708 154, EP-A-579 091, U.S. Pat. No. 5,411,586, U.S. Pat. No. 5,364,467, WO-A-97/39066, DE-A-4 225 031, WO 95/17479, and DE-A-196 14 637, the disclosures of all of which are hereby incorporated by reference.

The term “at least one filler” may be understood to include colorless and white, inorganic and synthetic and lamellar and nonlamellar particles. The term “at least one pearlescent agent” may be understood to include iridescent particles, for example produced by certain shellfish in their shells or else synthesized. The at least one filler and the at least one pearlescent agent may be used in certain embodiments to modify the texture of the composition.

The at least one pigment other than the at least one goniochromatic pigment according to the invention may be present in the composition in an amount, for example, up to 5%, in certain embodiments in an amount ranging from 0.01 to 5%, by weight with respect to the total weight of the composition. Obviously, the at least one other pigment will not be used in an amount that hides the goniochromatic effect of the at least one goniochromatic pigment according to the invention. The at least one pigment other than the at least one goniochromatic pigment according to the invention may be chosen from the examples mentioned below. Mention may be made, as inorganic pigments which can be used according to the invention, of titanium oxides, zirconium oxides, cerium oxides, zinc oxides, iron oxides, chromium oxides, ferric blue, and their mixtures. Mention may be made, among organic pigments which may be used according to the invention, of carbon black, barium lakes, strontium lakes, zirconium lakes, calcium lakes, aluminum lakes, the diketopyrrolopyrroles (DPPs) disclosed in documents EP-A-542 669, EP-A-787 730, EP-A-787 731, and WO 96/08537, the disclosures of all of which are hereby incorporated by reference, and their mixtures.

The at least one pearlescent agent may be present in the composition in an amount, for example, up to 20%, in certain embodiments in an amount ranging from 0.01 to 15%, by weight with respect to the total weight of the composition. For example, the at least one pearlescent agent which may be used in the invention is chosen from mica covered with titanium oxide, mica covered with iron oxide, mica covered with natural pigment, mica covered with bismuth oxychloride, such as colored titanium oxide-coated mica, and their mixtures.

The at least one filler may be present in the composition in an amount, for example, up to 50%, in certain embodiments in an amount ranging from 0.05 to 30%, by weight with respect to the total weight of the composition. For example, the at least one filler which may be used according to the present invention may be chosen from talc, zinc stearate, mica, kaolin, powders formed from nylon (in particular Orgasol powders) and from polyethylene, TEFLON®, starch, boron nitride, microspheres formed from copolymers, such as EXPANCEL (Nobel Industrie), POLYTRAP (Dow Corning), microspheres formed from poly(methyl methacrylate), silicone resin microbeads (TOSPEARL from Toshiba, for example), and their mixtures.

The composition may comprise a phase chosen from an aqueous phase, alcoholic phase, and an aqueous/alcoholic phase in a form dispersed or emulsified in the at least one continuous lipophilic phase. The at least one continuous lipophilic phase may thus comprise at least one substance chosen from at least one of water, alcohols, and acetone. In certain embodiments, said at least one substance is an alcohol chosen, for example, from linear and branched lower monoalcohols having from 2 to 5 carbon atoms, such as ethanol polyols and propanol, polyols, such as glycerol, diglycerol, propylene glycol, sorbitol, panthenol, pentylene glycol, polyethylene glycols, and their mixtures. The at least one substance may be present in the composition in an amount, for example, up to 70%, in certain embodiments in an amount ranging from 0.05 to 60%, by weight with respect to the total weight of the composition. The at least one substance may additionally comprise at least one hydrophilic (C₂) ether and at least one hydrophilic (C₂-C₄) aldehyde.

The composition according to the invention may be in a form chosen from cream, pomade, fluid lotion, soft paste with a dynamic viscosity at 25° C. of the order of 1 to 40 Pa·s, ointment, cast solid, and molded solid, in certain embodiments in a form chosen from a stick and in a dish.

The composition according to the invention may furthermore comprise at least one ingredient conventionally used in the fields under consideration and for example in the cosmetic and dermatological fields. The at least one ingredient may be chosen for example from preservatives, thickeners for an aqueous phase, such as polysaccharide biopolymers and synthetic polymers, thickeners for a fatty phase, fragrances, hydrophilic active principles, such as moisturizers, for example chosen from water and polyhydric alcohols having from 2 to 8 carbon atoms and from 2 to 6 hydroxyl functional groups, for example chosen from ethylene glycol, glycerol, 1,2-propanediol, diglycerol, erythritol, arabitol, adonitol, sorbitol, dulcitol and D-panthenol, and lipophilic active principles such as, for example chosen from lanolin, UV-A screening agents, and UV-B screening agents, antioxidants, colorants, essential oils, plant extracts, vitamins, vitamin derivatives, such as vitamins A, B, C and E, sphingolipids (ceramides), fat-soluble polymers, for example hydrocarbonaceous polymers such as polybutene, polyalkylenes, polyacrylates compatible with fatty substances, and silicone polymers compatible with fatty substances, and their mixtures. The amount of the at least one ingredient is an amount conventionally used in the fields under consideration. For example, the at least one ingredient may be present in the composition in an amount, for example, up to 20%, in certain embodiments in an amount ranging from 0.01 to 15%, by weight with respect to the total weight of the composition. The nature of the at least one ingredient and its proportion must be compatible with the composition according to the invention comprising at least one goniochromatic pigment to achieve at least a goniochromatic effect..

The composition according to the invention may be used for making up the skin and/or superficial body growths, i.e., the skin, the hair, the eyelashes, or the eyebrows of human beings, depending upon the nature of the constituents used to achieve the goniochromatic effect of the invention. In embodiments, this composition may be a lip lacquer or a lip gloss which may be used as such for applying a lipstick film, for providing at least a goniochromatic effect. The composition may also constitute at least one of a liquid foundation, a solid foundation, a concealer for the outline of the eyes, a product for the outlines of the eyes, an eyeliner, a mascara, and an eyeshadow. More specifically, an embodiment of the invention is chosen from a lip product, a foundation, and a mascara.

Another embodiment of the invention is the cosmetic use of the above composition for at least one of caring for, making up, and protecting the skin and the superficial body growths of human beings, i.e., in certain embodiments, the skin, the hair, the eyelashes, the lips, or the eyebrows of human beings, and the use of this composition for the preparation of an ointment intended to treat and/or protect the skin and the superficial body growths i.e., in certain embodiments, the skin, the hair, the eyelashes, the lips, or the eyebrows of human beings. A further embodiment of the invention is a process for the cosmetic treatment of the skin, the hair, the eyelashes, or the eyebrows, comprising applying to the skin and the superficial body growths i.e., in certain embodiments, the skin, the hair, the lips, the eyelashes, or the eyebrows of human beings, the composition according to the invention to achieve the goniochromatic effect.

The composition of the invention may be obtained according to the preparation processes conventionally used in cosmetics or in dermatology.

A further embodiment of the invention is a method for producing a goniochromatic effect on the skin and superficial body growths of human beings comprising applying to the skin and superficial body growths of human beings a composition comprising at least one goniochromatic pigment, and a cosmetically acceptable medium comprising at least one continuous lipophilic phase.

A further embodiment of the invention is a process for making up the skin and/or the superficial body growths of human beings i.e., in certain embodiments, the skin, the hair, the lips, the eyelashes, or the eyebrows of human beings, comprising applying to the skin, the hair, the eyelashes, the lips, or the eyebrows, the composition to achieve a goniochromatic effect according to the invention.

Certain embodiments of the invention are illustrated below and are not intended to be limiting in nature. As used herein, the phrase “at least one” means one or more.

### EXAMPLE 1

### Anhydrous Foundation

A foundation was prepared in the form of an anhydrous stick having the following composition:

| | | |
|---|---|---|
| Isononyl isononanoate | | 15% |
| 2-Ethylhexyl palmitate | q.s. for | 100% |
| Silicone oil (polydimethylsiloxane) | | 17% |
| Waxes | | 6% |
| Interferential goniochromatic pigment: | | |
| SICOPEARL FANTASTICO OR from BASF | | 10% |
| Nylon powder | | 16% |
| PTFE wax | | 7% |
| Silicone | | 8% |

A foundation was obtained which had a yellow/green color and which exhibited a goniochromatic effect once applied to the skin, for example the face.

In the same way, by replacing the at least one goniochromatic pigment SICOPEARL FANTASTICO OR with at least one goniochromatic pigment SICOPEARL FANTASTICO ROSE from BASF, a foundation was obtained which had a pink/brown-pink color and which exhibited a goniochromatic effect once applied to the skin, for example the face.

In the same way, by replacing the interferential goniochromatic pigment SICOPEARL FANTASTICO OR or ROSE with at least one goniochromatic pigment SICOPEARL FANTASTICO RUBY from BASF, a foundation was obtained which had a red/orange color and which exhibited a goniochromatic effect once applied to the skin, for example the face.

### EXAMPLE 2

### Eyeliner

An eyeliner was prepared having the following composition:

| | | |
|---|---|---|
| Waxes | | 6.9 g |
| Interferential goniochromatic pigment: | | |
| SICOPEARL FANTASTICO RUBY from BASF | | 10 g |
| Thickening agent | | 7 g |
| PLIOWAY ® Ultra 200 copolymer | | 10 g |
| Vinyl acetate/allyl stearate (65/35) copolymer | | 6 g |
| (fat-soluble polymer) | | |
| Rice starch | | 0.95 g |
| Light paraffinic and naphthenic hydrocarbons | q.s. for | 100 g |
| (SHELL SOLT from Shell) | | |

An anhydrous and waterproof eyeliner was obtained which was black in color, with yellow to green highlights, depending upon the angle of the light, and which exhibited a goniochromatic effect.

### EXAMPLE 3

### Mascara

A mascara was prepared having the following composition:

| | |
|---|---|
| Paraffin wax | 2 g |
| Carnauba wax | 4.2 g |
| Beeswax | 7.4 g |
| Poly(vinyl laurate) (Mexomère PP from Chimex) | 0.66 g |
| Vinyl acetate/allyl stearate (65/35) copolymer | 1.96 g |
| PLIOWAY ® Ultra G20 copolymer from Goodyear | 10 g |
| Polymer as an aqueous dispersion* | 2.5 g AM |
| Rice starch | 0.74 g |
| Interferential goniochromatic pigment: | 5 g |
| SICOPEARL FANTASTICO RUBY from BASF | |
| Preservatives | q.s. |
| Isododecane | q.s. for 100 g |

*Vinyl acetate/crotonic acid/vinyl 4-(tert-butyl)benzoate (65/10/25) copolymer, 65% neutralized with 2-amino-2-methyl-1-propanol and 25% plasticized with diisopropyl adipate, prepared according to the teaching of the document EP-A-655 234, the disclosure of which is hereby incorporated by reference.

The nonanhydrous mascara was easily applied to the eyelashes. When the composition according to the invention was applied directly to black eyelashes, a very intense green makeup was obtained which became blue according to the angle of observation.

### EXAMPLE 4

### Lipstick

A solid dispersion was prepared in the form of a lipstick stick according to European patent application EP-A1-0 524 892, the disclosure of which is hereby incorporated by reference, which dispersion has the following composition (as weight %):

| | |
|---|---|
| Castor oil | 3% |
| Liquid petrolatum | 9% |
| Lanolin | 15% |
| Butylated hydroxytoluene (BHT) | 0.2% |
| Beeswax | 8.8% |
| Ozokerite | 10% |
| Soybean lecithin | 4% |
| Glycerol | 10% |
| Polyethylene glycol 300 | 12% |
| Talc | 3% |
| D & C Red 27 | 5% |
| D & C Red 7 | 6% |
| SICOPEARL FANTASTICO OR pigment from BASF | 2% |
| Polybutene (INDOPOL from Amoco) | 12% |
| Fragrance q.s. | |

A lipstick was obtained which was yellow to green in color, depending upon the angle of the light, and which exhibited a goniochromatic effect once applied to the lips.

In the same way, by replacing the at least one goniochromatic pigment SICOPEARL FANTASTICO ROSE with at least one goniochromatic pigment SICOPEARL FANTASTICO RUBY from BASF, a lipstick was obtained which was pink to brown-pink in color, depending upon the angle of the light and the angle of observation, and which exhibited a goniochromatic effect once applied to the lips.

### EXAMPLE 5

### W/O Foundation

A foundation of water-in-oil type was prepared having the following composition:

| | | |
|---|---|---|
| Surfactant sold under the trade name “ABIL | | 6% |
| WE 09” by Goldschmidt | | |
| Isostearyl diglyceryl succinate, sold under | | 2% |
| the trade name “IMWITOR 780 K” by Hüls | | |
| Cyclomethicone | | 25% |
| Isododecane | | 4.55% |
| Interferential goniochromatic pigment: | | |
| SICOPEARL FANTASTICO OR from BASF | | 10% |
| Nylon powder | | 8% |
| Vinyl acetate/vinyl p-(tert-butyl)benzoate/ | | 20% |
| crotonic acid copolymer, partially neutralized | | |
| and stabilized, as an aqueous dispersion | | |
| Diisopropyl adipate | | 1% |
| Water | q.s. for | 100% |

A foundation was obtained which was yellow/green in color and which exhibited a goniochromatic effect once applied to the skin, for example of the face.

In the same way, by replacing the at least one goniochromatic pigment SICOPEARL FANTASTICO OR with at least one goniochromatic pigment SICOPEARL FANTASTICO ROSE from BASF, a foundation was obtained which was pink/brown-pink in color and which exhibited a goniochromatic effect once applied to the skin, for example the face.

In the same way, by replacing the at least one goniochromatic pigment SICOPEARL FANTASTICO OR or ROSE with at least one goniochromatic pigment SICOPEARL FANTASTICO RUBY from BASF, a foundation was obtained which was red/orange in color and which exhibited a goniochromatic effect once applied to the skin, for example the face.

What is claimed is:

1. A method for producing a goniochromatic effect on skin, hair, eyelashes, or eyebrows of human beings comprising:
applying to the skin, hair, eyelashes, or eyebrows of human beings a cosmetic composition comprising at least one continuous lipophilic phase and at least one goniochromatic pigment.

2. The method as claimed in claim 1, wherein the cosmetic composition further comprises a cosmetically acceptable medium.

3. The method as claimed in claim 1, wherein the at least one goniochromatic pigment has an interferential multilayer structure comprising at least two layers, each layer being made of at least one material chosen from MgF₂, CeF₃, ZnS, ZnSe, Si, SiO₂, Ge, Te, Fe₂O₃, Pt, V, Al₂O₃, MgO, Y₂O₃, S₂O₃, SiO, HfO₂, ZrO₂, CeO₂, Nb₂O₅, Ta₂O₅, TiO₂, Ag, Al, Au, Cu, Rb, Tl, Ta, W, Zn, MoS₂, Cr, mica oxide, and cryolite.

4. The method as claimed in claim 3, wherein the interferential multilayer structure is chosen from: Al/SiO₂/Al/SiO₂/Al; Cr/MgF₂/Al/MgF₂/Al; MoS₂/SiO₂/Al/SiO₂/MoS₂; Fe₂O₃/SiO₂/Al/SiO₂/Fe₂O₃; Fe₂O₃/SiO₂/Fe₂O₃/SiO₂/Fe₂O₃; MoS₂/SiO₂/mica-oxide/SiO₂/MoS₂; and Fe₂O₃/SiO₂/mica-oxide/SiO₂/Fe₂O₃.

5. The method as claimed in claim 1, wherein said at least one goniochromatic pigment is present in the composition in an amount ranging from 0.01 to 50% by weight with respect to the total weight of said composition.

6. The method as claimed in claim 5, wherein said at least one goniochromatic pigment is present in the composition in an amount ranging from 0.5 to 25% by weight with respect to the total weight of the said composition.

7. The method as claimed in claim 1, wherein the compostion is provided in a form of a product to be applied to the skin, the hair, the eyelashes, or the eyebrows of human beings.

8. The method as claimed in claim 2, wherein said cosmetically acceptable medium comprises or is provided in a form chosen from a suspension in water, a dispersion in water, a solution in water, an aqueous/alcoholic medium, oil-in-water (O/W) emulsion, multiple (W/O/W) emulsion, aqueous gel, aqueous/alcoholic gel, hydrophilic foam, emulsified gel, dispersion of vesicles, two-phase lotion, multiphase lotion, and spray.

9. The method as claimed in claim 2, wherein said cosmetically acceptable medium comprises or is provided in a form chosen from an oil-in-water (O/W) emulsion and a multiple (W/O/W) emulsion.

10. The method as claimed in claim 1, wherein said composition further comprises at least one surfactant present in the composition in an amount up to 30% by weight with respect to the total weight of said composition.

11. The method as claimed in claim 10, wherein the at least one surfactant is present in the composition in an amount ranging from 0.5 to 25% by weight with respect to the total weight of the composition.

12. The method as claimed in claim 1, wherein the at least one continuous lipophilic phase is present in the composition in an amount ranging from 0.5 to 99.99% by weight with respect to the total weight of the composition.

13. The method as claimed in claim 2, wherein said cosmetically acceptable medium comprises at least one lipophilic substance chosen from at least one fatty substance which is liquid at ambient temperature and atmospheric pressure, at least one water-immiscible organic solvent, and at least one fatty substance which is solid at ambient temperature and at atmospheric pressure.

14. The method as claimed in claim 13, wherein said at least one fatty substance which is liquid at ambient temperature and at atmospheric pressure is chosen from hydrocarbonaceous oils of animal origin; vegetable hydrocarbonaceous oils; linear and branched hydrocarbons; liquid paraffins and their derivatives; synthetic esters and ethers; polyol esters; fatty alcohols having from 12 to 26 carbon atoms; heptanoates, octanoates of fatty alcohols; decanoates of fatty alcohols; fluorinated oils which are chosen from partially hydrocarbon-comprising and silicone-comprising; and silicone oils.

15. The method as claimed in claim 14, wherein the silicone oils are chosen from polydimethylsiloxanes (PDMSs).

16. The method as claimed in claim 13, wherein said at least one fatty substance which is liquid at ambient temperature and at atmospheric pressure is chosen from perhydrosqualene, triglycerides of heptanoic acid, triglycerides of octanoic acid, sunflower oil, maize oil, soybean oil, grape seed oil, sesame oil, apricot oil, macadamia oil, castor oil, and avocado oil, triglycerides of caprylic/capric acids, jojoba oil, karite butter oil, liquid petrolatum, polydecenes, hydrogenated polyisobutene, isododecane, Purcellin oil, isopropyl myristate, 2-ethylhexyl palmitate, 2-octyldodecyl stearate, 2-octyidodecyl erucate, isostearyl isostearate, isononyl isononanoate, isostearyl lactate, octyl hydroxystearate, octyldodecyl hydroxystearate, diisostearyl malate, triisocetyl citrate, propylene glycol dioctanoate, neopentyl glycol diheptanoate, diethylene glycol diisononanoate, pentaerythritol esters, octydodecanol, 2-butyloctanol, 2-hexyldecanol, 2-undecylpentadecanol, oleyl alcohol, cyclomethicones, dimethicones, phenyl trimethicones, phenyl trimethylsiloxydiphenylsiloxanes, diphenylmethyldimethyltrisiloxanes, diphenyl dimethicones, phenyl dimethicones, and polymethylphenylsiloxanes.

17. The method as claimed in claim 13, wherein said at least one fatty substance which is liquid at ambient temperature and at atmospheric pressure is present in the at least one continuous lipophilic phase in an amount up to 90% by weight with respect to the total weight of the at least one continuous lipophilic phase.

18. The method as claimed in claim 13, wherein said at least one fatty substance which is liquid at ambient temperature and at atmospheric pressure is present in the at least one continuous lipophilic phase in an amount ranging from 0.01 to 85% by weight with respect to the total weight of the at least one continuous lipophilic phase.

19. The method as claimed in claim 13, wherein said at least one fatty substance which is solid at ambient temperature and at atmospheric pressure is chosen from at least one wax, at least one pasty fatty substance, and at least one gum.

20. The method as claimed in claim 13, wherein said at least one fatty substance which is solid at ambient temperature and at atmospheric pressure is present in the composition in an amount up to 50% by weight with respect to the total weight of the composition.

21. The method as claimed in claim 13, wherein said at least one fatty substance which is solid at ambient temperature and at atmospheric pressure is present in the composition in an amount ranging from 0.01 to 40% by weight with respect to the total weight of the composition.

22. The method as claimed in claim 13, wherein said at least one fatty substance which is solid at ambient temperature and at atmospheric pressure is present in the composition in an amount ranging from 0.1 to 30% by weight with respect to the total weight of the composition.

23. The method as claimed in claim 18, wherein said at least one fatty substance which is solid at ambient temperature and at atmospheric pressure is chosen from beeswax, carnauba wax, candelilla wax, paraffin wax, microcrystalline waxes, ceresin, ozokerite, synthetic waxes, silicone waxes, PDMSs with a high molecular weight, cellulose gums, and polysaccharides.

24. The method as claimed in claim 1, wherein the composition further comprises at least one film-forming polymer.

25. The method as claimed in claim 1, wherein the composition further comprises at least one additional particulate phase in an amount up to 50% by weight with respect to the total weight of said composition.

26. The method as claimed in claim 25, wherein the at least one additional particulate phase is present in the composition in an amount ranging from 0.01 to 40% by weight with respect to the total weight of said composition.

27. The method as claimed in claim 25, wherein the at least one additional particulate phase is present in the composition in an amount ranging from 0.05 to 30% by weight with respect to the total weight of said composition.

28. The method as claimed in claim 25, wherein said at least one additional particulate phase comprises at least one pigment other than the at least one goniochromatic pigment.

29. The method as claimed in claim 25, wherein said at least one additional particulate phase comprises at least one pearlescent agent.

30. The method as claimed in claim 25, wherein said at least one additional particulate phase comprises at least one filler.

31. The method as claimed in claim 28, wherein the at least one pigment other than the at least one goniochromatic pigment is present in the composition in an amount up to 5% by weight with respect to the total weight of the composition.

32. The method as claimed in claim 28, wherein the at least one pigment other than the at least one goniochromatic pigment is present in the composition in an amount ranging from 0.01 to 5% by weight with respect to the total weight of the composition.

33. The method as claimed in claim 28, wherein said at least one pigment other than the at least one goniochromatic pigment is chosen from titanium oxides, zirconium oxides, cerium oxides, zinc oxides, chromium oxides, ferric blue, carbon black, barium, strontium, zirconium, calcium lakes, aluminum lakes, and diketopyrrolopyrroles (DPPs).

34. The method as claimed in claim 29, wherein the at least one pearlescent agent is present in the composition in an amount up to 20% by weight with respect to the total weight of the composition.

35. The method as claimed in claim 29, wherein the at least one pearlescent agent is present in the composition in an amount ranging from 0.01 to 15% by weight with respect to the total weight of the composition.

36. The method as claimed in claim 29, wherein the at least one pearlescent agent is chosen from mica covered with titanium oxide, mica covered with iron oxide, mica covered with natural pigment, and mica covered with bismuth oxychloride.

37. The method as claimed in claim 30, wherein the at least one filler is present in the composition in an amount up to 50% by weight with respect to the total weight of the composition.

38. The method as claimed in claim 30, wherein the at least one filler is present in the composition in an amount ranging from 0.05 to 30% by weight with respect to the total weight of the composition.

39. The method as claimed in claim 30, wherein the at least one filler is chosen from talc, zinc stearate, mica, kaolin, powders formed from nylon, powders formed from polyethylene, Teflon, starch, boron nitride, microspheres formed from copolymers, Polytrap, and silicone resin microbeads.

40. The method as claimed in claim 1, wherein the at least one continuous lipophilic phase comprises at least one substance chosen from linear and branched lower monoalcohols having from 2 to 5 carbon atoms, and polyols.

41. The method as claimed in claim 40, wherein the least one substance is chosen from ethanol, propanol, glycerol, diglycerol, propylene glycol, sorbitol, panthenol, pentylene glycol, and polyethylene glycols.

42. The method as claimed in claim 40, wherein said at least one substance is additionally chosen from at least one hydrophilic (C₂) ether and at least one hydrophilic (C₂-C₄) aldehyde.

43. The method as claimed in claim 1, wherein the composition further comprises at least one additional ingredient in an amount up to 20% by weight with respect to the total weight of the composition.

44. The method as claimed in claim 43, wherein the at least one additional ingredient is present in the composition in an amount ranging from 0.01 to 15% by weight with respect to the total weight of the composition.

45. The method as claimed in claim 43, wherein the at least one additional ingredient is chosen from preservatives, thickeners for an aqueous phase, thickeners for a fatty phase, fragrances, hydrophilic active principles, lipophilic active principles, antioxidants, colorants, essential oils, plant extracts, vitamins and their derivatives, sphingolipids, fat-soluble polymers, and their mixtures.

46. The method as claimed in claim 43, wherein the at least one additional ingredient is chosen from ethylene glycol, glycerol, 1,2-propanediol, diglycerol, erythritol, arabitol, adonitol, sorbitol, dulcitol, D-panthenol, lanolin, UV-A screening agents, UV-B screening agents, vitamins A, B, C, and E, and their derivatives, polybutene, polyalkylenes, polyacrylates polymers compatible with fatty substances, and silicone polymers compatible with fatty substances.

47. The method as claimed in claim 1, wherein the composition is chosen from liquid foundations, solid foundations, concealers for the outline of the eyes, products for the outline of the eyes, eyeliners, mascaras, and eyeshadows.

48. A method for preparing an ointment which exhibits a goniochromatic effect when applied to skin, hair, eyelashes, and eyebrows of human beings comprising:
including at least one goniochromatic pigment in a cosmetic composition comprising at least one continuous lipophilic phase.
